(19) United States
(12) Patent Application Publication
(10) Pub. No.: US 2025/0230221 A1
(43) Pub. Date: Jul. 17, 2025

(54) OPTIMIZING POLYPEPTIDE SIALIC ACID CONTENT

(71) Applicant: GENENTECH, INC., South San Francisco, CA (US)

(72) Inventors: NATARAJAN VIJAYASANKARAN, South San Francisco, CA (US); KYLE DAVID HIRST, South San Francisco, CA (US)

(73) Assignee: GENENTECH, INC., South San Francisco, CA (US)

(21) Appl. No.: 19/098,734

(22) Filed: Apr. 2, 2025

Related U.S. Application Data
(63) Continuation of application No. PCT/US23/83382, filed on Dec. 11, 2023.
(60) Provisional application No. 63/431,945, filed on Dec. 12, 2022.

(51) Int. Cl. C07K 16/00

(52) U.S. Cl. CPC C07K 16/00; C07K 2317/14; C07K 2317/41

(57) ABSTRACT

We provide a method of optimizing sialic acid content for a recombinant polypeptide. The method comprises culturing a cell-line engineered to express the recombinant polypeptide under conditions promoting production of the recombinant polypeptide; monitoring cell viability of the cultured cell-line on at least one of days 7, 8, 9, 10, 11 or 12 of the culturing. If the cell viability is at or below a threshold level, the method also comprises stopping the culture and isolating the recombinant polypeptide after a first predetermined additional time. If the cell viability is above the threshold level, the method also comprises stopping the culture and isolating the recombinant polypeptide after a second predetermined additional time. The second predetermined additional time is at least about 12 hours longer than the first predetermined time.

### CROSS-REFERENCE TO RELATED APPLICATIONS

This application claims priority to U.S. Provisional Application No. 63/431,945 filed Dec. 12, 2022, the contents of which are incorporated by reference in their entirety.

This disclosure relates to methods of optimizing sialic acid content for recombinant polypeptides.

### BACKGROUND

Commercially important proteins, such as therapeutic proteins, enzymes and antibodies, may be made via cell culture. In such a cell culture, a host cell line comprises genetic material that encodes the protein of interest. Under ideal conditions, the host cell line will express the proteins of interest. Variation in post-translational modification of the expressed protein can, however, have an effect on the quality of the protein that is produced. This is particularly important for biopharmaceutical products, where the specification of the product will typically specify quality attributes that are related to post translational modifications.

Many important biopharmaceutical protein products are typically produced by performing cell culture in bioreactors and purifying the desired product from the extracellular medium. Cell culture processes utilize living cells and typically require highly skilled personnel, techniques must be performed using strict aseptic techniques because animal cells grow slower than many of the common contaminants (e.g., bacteria, viruses and fungi). In addition, relatively modest changes to the cells or the culturing conditions may result in reduction in the quality of the protein product. An example of an important product quality related post translational modification is the level of sialic acid present in the final protein/polypepetide obtained. Direct analysis of the level of sialic acid in expressed protein is challenging and time consuming.

It is therefore desirable to provide improved and/or more timely methods for optimizing the level of sialic acid in a recombinant polypeptide/protein product.

### BRIEF SUMMARY OF THE DISCLOSURE

There remains a need to provide methods for the optimization of the sialic acid content for recombinant polypeptides. “Optimization” in this context means obtaining a recombinant polypeptide product, e.g. a therapeutic protein (such as an antibody or antibody related polypeptide) with the level sialic acid per molecule at a desired level. For example, many recombinant polypeptides (such as therapeutic proteins) will have a specification that requires sialic acid level to be at a specified level, in which case optimization comprises having the sialic acid content of the recombinant polypeptide within the specification.

In methods used to culture recombinant protein, the level of sialic acid content often decreases with culture duration. Such a decrease may result is a recombinant polypeptide product that has a sub-optimal sialic acid level. For example, it may result in a therapeutic that cannot be used, as its sialic acid level is below the specification. The time point can, however, vary from culture to culture, even when the same cell culture method is used due to inherent process variability. This means that it can be difficult to obtain a recombinant polypeptide product with an optimal level of sialic acid, especially while also looking to obtain a high titer of the product.

We have noted that cell viability is predictive of the level of sialic acid. In particular, we have determined that cell viability as determined at a relatively early stage of the cell culture (e.g. on one of days 6 to 11) is predictive of optimal level of sialic acid at a relatively later stage of the cell culture (e.g. on one or more of days 9 to 14). In view of this, we have developed exemplary methods of the present invention.

In accordance with the present invention there is provided a method of optimizing sialic acid content for a recombinant polypeptide. The method comprises culturing a cell-line engineered to express the recombinant polypeptide under conditions promoting production of the recombinant polypeptide: monitoring cell viability of the cultured cell-line on at least one of days 6, 7, 8, 9, 10, 11 or 12 of the culturing. If the cell viability is at or below a threshold level, the method also comprises stopping the culture and isolating the recombinant polypeptide after a first predetermined additional time. If the cell viability is above the threshold level, the method also comprises stopping the culture and isolating the recombinant polypeptide after a second predetermined additional time. The second predetermined additional time is at least about 10 hours longer than the first predetermined time.

### BRIEF DESCRIPTION OF THE DRAWINGS

Embodiments of the invention are further described hereinafter with reference to the accompanying drawings, in which:

FIG. 1 provides an illustration of the level of sialic acid for a recombinant Fc fusion protein obtained from a cell culture from about 9 to about 14 days of cell culture. The Fc fusion protein had eight N-linked glycosylation sites. The cell culture comprises a CHO cell line that was engineered to express the Fc fusion protein. As illustrated in the figure, the level of sialic acid, in mol sialic acid per mol recombinant protein, decreased over time.

FIG. 2 illustrates a typical progression of an exemplary abundant glycan form for a recombinant Fc fusion protein obtained from a cell culture from about 9 to about 14 days of cell culture. The circles represent galactose (Gal), the pentagons represent mannose (Man), the squares represent N-acetylglucosamine (GlcNAc), the triangles represent fucose (Fuc), and the diamonds represent sialic acid (NANA).

FIG. 3 demonstrates a general correlation that was identified the viability of the cells in the culture and level of sialic acid.

FIG. 4 illustrates a correlation between the viability of cells in the cell culture on days 6, 7, 8, 9, 10, and 11 and the level of sialic acid on day 12.

FIG. 5 illustrates the correlation between the viability measured on days 8, 9, 10 of the culture and the levels of a sialylated glycan species (G2-1SA) at the end of the culture (day 12). G2-1SA is normalized vs the highest reported G2-1SA in the sample set of optimally sialylated antibody (i.e. normalized G2-1SA of 0.50 is 50% of the highest reported G2-1SA value in the data set).

FIG. 6 provides an illustration of the correlation between normalized G2-ISA and culture duration, showing that viability measurements earlier in the culture may be used to control the levels of sialylation at the end of the culture, by changing the culture duration based on the earlier viability measurement.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

All publications, patent applications, patents, and other references mentioned herein are incorporated by reference in their entirety. In case of conflict, the present specification, including definitions, will control.

### Definitions

The following explanations of terms and methods are provided to better describe the present disclosure and to guide those of ordinary skill in the art in the practice of the present disclosure.

The term “about” or “approximately”, unless otherwise stated, means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “polypeptide” and “protein” can be used interchangeably and refer generally to peptides and proteins having more than about 10 covalently attached amino acids linked by a peptidyl bond. The term protein encompasses purified natural products, or products which may be produced partially or wholly using recombinant or synthetic techniques. The terms peptide and protein may refer to an aggregate of a protein such as a dimer or other multimer, a fusion protein, a protein variant, or derivative thereof. The term also includes modifications of the protein, for example, protein modified by glycosylation, acetylation, phosphorylation,

PEGylation, ubiquitination, and so forth. A protein may comprise amino acids not encoded by a nucleic acid codon. A protein may have a sequence of amino acids of sufficient length to produce higher levels of tertiary and/or quaternary structure. A typical protein herein may have a molecular weight of at least about 15-20 kDa, preferably at least about 20 kDa. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “post translational modifications” (PTMs) means, unless otherwise stated, covalent modifications of proteins following protein biosynthesis. Exemplary PTMs that may be determined by methods of the invention (or determined using systems of the invention) include glycosylation, deamidation, succinimidation, isomerization, C-terminal clipping, N-terminal cyclization, oxidation, and proteolysis.

The term “antibody”, unless otherwise stated, encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies, for example as disclosed in Labrijn, et al., “Bispecific antibodies: a mechanistic review of the pipeline”, Nat. Rev. Drug Discov., 2019 Aug. 18(8), 585-608) and antibody fragments so long as they exhibit the desired antigen-binding activity. A therapeutic antibody is an antibody that may be used in the treatment of a disease.

The terms “antibody fragment”, “antigen-binding portion” of an antibody (or simply “antibody portion”) or “antigen-binding fragment” of an antibody, unless otherwise stated, refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂: diabodies: linear antibodies: single-chain antibody molecules (e.g., scFv, and scFab): single domain antibodies (dAbs); and multispecific antibodies formed from antibody fragments. For a review of certain antibody fragments, see Holliger and Hudson, Nature Biotechnology 23:1126-1136 (2005). A therapeutic antibody fragment is an antibody fragment that may be used in the treatment of a disease.

The term “chimeric” antibody means an antibody in which a portion of the heavy and/or light chain is derived from a particular source or species, while the remainder of the heavy and/or light chain is derived from a different source or species.

The term “human antibody”, unless otherwise stated, refer to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody”, unless otherwise stated, refer to a chimeric antibody comprising amino acid residues from non-human CDRs and amino acid residues from human FRs. In examples, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “monoclonal antibody”, unless otherwise stated, refer to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “isolated” means a biological component (such as a nucleic acid molecule, polypeptide, or protein) that has been substantially separated or purified away from other biological components in the cell of the organism in which the component naturally occurs, i.e., other chromosomal and extrachromosomal DNA and RNA, and proteins. Nucleic acids, polypeptides and proteins that have been “isolated” include nucleic acids, polypeptides and proteins purified by standard purification methods. The term also embraces nucleic acids, polypeptides and proteins prepared by recombinant expression in a host cell as well as chemically synthesized nucleic acids, polypeptides and proteins. An isolated component may be purified.

Purified: The term purified does not require absolute purity: rather, it is intended as a relative term. Thus, for example, a purified product is one in which the product is more enriched than the product (e.g. polypeptide or protein) is in its environment within a cell, such that the product is substantially separated from cellular components (nucleic acids, lipids, carbohydrates, and [other] polypeptides) that may accompany it.

Purified: The term purified does not require absolute purity: rather, it is intended as a relative term. Thus, for example, a purified product is one in which the product is more enriched than the product (e.g. polypeptide or protein) is in its environment within a cell, such that the product is substantially separated from cellular components (nucleic acids, lipids, carbohydrates, and [other] polypeptides) that may accompany it.

In one example, a product of interest of the disclosure (e.g. a recombinant polypeptide) is purified when at least 50% by weight of a sample is composed of the product, for example when at least 60%, 70%, 80%, 85%, 90%, 92%, 95%, 98%, or 99% or more of a sample is composed of the polypeptide. Examples of methods that can be used to purify a polypeptide, include, but are not limited to the methods disclosed in Sambrook et al. (Molecular Cloning: A Laboratory Manual, Cold Spring Harbor, N.Y., 1989, Ch. 17). Protein purity can be determined by, for example, high-pressure liquid chromatography or other conventional methods.

The term “titer” means the total amount of a product of interest (e.g. a recombinant polypeptide, such as an antibody) produced by a cell culture divided by a given amount of medium volume. Titer is typically expressed in units of milligrams of antibody per milliliter or liter of medium (mg/ml or mg/L). In certain embodiments, titer is expressed in grams of polypeptide per liter of medium (g/L). Titer can be expressed or assessed in terms of a relative measurement, such as a percentage increase in titer as compared obtaining the protein product under different culture conditions.

The term “cell” as used herein includes reference to a eukaryotic cell. Unless the context requires otherwise, reference to a cell may include reference to the plural (cells). A eukaryotic cell may be an animal cell (e.g. a mammalian cell). A eukaryotic cell may be a mammalian cell, such as a hybridoma, CHO cell, COS cell, VERO cell, HeLa cell, HEK 293 cell, PER-C6 cell, K562 cell, MOLT-4 cell, MI cell, NSO cell, NS-1 cell, COS-7 cell, MDBK cell, MDCK cell, MRC-5 cell, WI-38 cell, WEHI cell, SP2/0 cell, BHK cell (including BHK-21 cell) and derivatives thereof. A cell may be a CHO cell, an NSO cell, or a derivative thereof. A CHO cell may be, for example, a CHO K1 cell, a CHO K1SV cell, a DG44 cell, a DUKXB-11 cell, a CHOKIS cell, a CHO K1M cell, a targeted gene integration (TI)-generated CHO cell line, and derivatives thereof.

The term “cell line” as used herein includes reference to a culture of eukaryotic cells that can be propagated repeatedly. The eukaryotic cells of the cell line may be selected from any cell as defined herein.

The terms “host cell,” “host cell line” and “host cell culture” are used interchangeably herein to refer to cells into which exogenous nucleic acid has been introduced, including the progeny of such cells. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein.

The terms “mammalian host cell” or “mammalian cell” as used herein refer to cells and cell lines derived from mammals that are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. Typically, the cells are capable of expressing and secreting large quantities of a particular protein, e.g., glycoprotein, of interest into the culture medium. Examples of suitable mammalian host cells include Chinese hamster ovary cells/-DHFR (CHO, Urlaub and Chasin, Proc. Natl. Acad. Sci. USA, 77:4216 1980): dp12.CHO cells (EP 307,247 published 15 Mar. 1989): CHO-K1 (ATCC, CCL-61); monkey kidney CVI line transformed by SV40 (COS-7, ATCC CRL 1651); human embryonic kidney line (293 or 293 cells subcloned for growth in suspension culture, Graham et al., J. Gen Virol., 36:59 1977): baby hamster kidney cells (BHK, ATCC CCL 10): mouse sertoli cells (TM4, Mather, Biol. Reprod., 23:243-251 1980): monkey kidney cells (CVI ATCC CCL 70): African green monkey kidney cells (VERO-76, ATCC CRL-1587): human cervical carcinoma cells (HELA, ATCC CCL 2): canine kidney cells (MDCK, ATCC CCL 34): buffalo rat liver cells (BRL 3A, ATCC CRL 1442): human lung cells (W138, ATCC CCL 75): human liver cells (Hep G2, HB 8065): mouse mammary tumor (MMT 060562, ATCC CCL51): TRI cells (Mather et al., Annals N.Y. Acad. Sci., 383:44-68 1982): MRC 5 cells: FS4 cells; and a human hepatoma line (Hep G2). The mammalian cells may include Chinese hamster ovary cells/-DHFR (CHO, Urlaub and Chasin, Proc. Natl. Acad. Sci. USA, 77:4216 1980): dp12.CHO cells (EP 307,247 published 15 Mar. 1989).

The term “cell culture medium” as used herein refers to a nutritive solution for cultivating cells. A “cell culture feed” and a “cell culture additive” represent nutritive supplements that may be added to a cell culture medium to improve medium performance. For example, a cell culture feed and/or a cell culture additive may be added to a cell culture medium during batch culture of cells. A cell culture medium may be chemically defined or may comprise undefined components. Cell culture medium, for example for mammalian cells, typically comprises at least one component from one or more of the following categories:
1) an energy source, usually in the form of a carbohydrate such as glucose:
2) all essential amino acids, and usually the basic set of twenty amino acids plus cysteine;
3) vitamins and/or other organic compounds required at low concentrations:
4) free fatty acids; and
5) trace elements, where trace elements are defined as inorganic compounds or naturally occurring elements that are typically required at very low concentrations, usually in the micromolar range.

Cell culture media and similar nutrient solutions may optionally be supplemented with one or more components from any of the following categories:
1) hormones and other growth factors as, for example, insulin, transferrin, and epidermal growth factor:
2) salts and buffers as, for example, calcium, magnesium, and phosphate:
3) nucleosides and bases such as, for example, adenosine, thymidine, and hypoxanthine; and
4) protein and tissue hydrolysates.

The term “culturing” refers to contacting a cell or cells with a cell culture medium under conditions suitable to the survival and/or growth and/or proliferation of the cell. A “cell culture” refers to a cell or cells in contact with a cell culture medium.

The term “batch culture” refers to a culture in which all components for cell culturing (including the cells and all culture nutrients) are supplied to the culturing bioreactor at the start of the culturing process.

The term “fed batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.”

The term “perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.”

The terms “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene may be quantified by northern hybridization. Sambrook et al., Molecular Cloning: A Laboratory Manual, pp. 7.3-7.57 (Cold Spring Harbor Laboratory Press, 1989). Protein encoded by a product gene can be quantified either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

### Cell Culture

A cell culture comprises a cell culture medium and at least one (typically a plurality of) cells. For example, a cell culture medium may comprise a cell culture medium and a plurality of eukaryotic cells, for example cells that have been engineered to produce a protein product.

Cell culture media contain many components. Cell culture media provide the nutrients necessary to maintain and grow cells in a controlled, artificial and in vitro environment. Characteristics and compositions of the cell culture media vary depending on the particular cellular requirements. Important parameters include osmolarity, pH, and nutrient formulations.

Culture media contain a mixture of amino acids, glucose, salts, vitamins, and other nutrients, and are available either as a powder or as a liquid form from commercial suppliers. The requirements for these components vary among cell lines. Regulation of pH is critical for optimum culture conditions and is generally achieved using a suitable buffering system. While chemically defined media (CDM) are preferred for therapeutic and related applications, as CDM provide reproducible contamination-free media when prepared and used under aseptic conditions, for some cell types it may be necessary to use media comprising serum, proteins or other biological extracts (such as yeast extracts or enzymatic digests of plant or animal matter).

Some extremely simple defined media, which consist essentially of vitamins, amino acids, organic and inorganic salts and buffers have been used for cell culture. Such media (often called “basal media”), however, are usually seriously deficient in the nutritional content required by most animal cells. These media therefore often need to be supplemented, for example with feeds or other additives, to form complete media. In addition, batch culture systems often include periodic supplementation of the media with concentrated feeds or additives, to maintain the viability of cultured cells and/or production of biological products, such as polypeptides (e.g. antibodies, or biologically functional fragments of antibodies), proteins, peptides, hormones, viruses or virus like particles, nucleic acids or fragments thereof.

Ingredients that may be present in basal media include amino acids (nitrogen source), vitamins, inorganic salts, sugars (carbon source), buffering salts and lipids. Basal media for use with some mammalian cell culture systems may contain ethanolamine, D-glucose, N-[2-hydroxyethyl]-piperazine-N′-[2-ethanesulfonic acid] (HEPES), linoleic acid, lipoic acid, phenol red, PLURONIC F68, putrescine, sodium pyruvate.

Amino acid ingredients which may be included in the media include L-alanine, L-arginine, L-asparagine, L-aspartic acid, L-cysteine, L-glutamic acid, L glutamine, glycine, L-histidine, L-isoleucine, L-leucine, L-lysine, L-methionine, L phenylalanine, L-proline, L-serine, L-threonine, L-tryptophan, L-tyrosine, L-valine, and derivatives thereof. These amino acids may be obtained commercially, for example from Sigma (Saint Louis, Missouri).

Vitamin ingredients which may be included in the media include biotin, choline chloride, D-Ca²⁺-pantothenate, folic acid, i-inositol, niacinamide, pyridoxine, riboflavin, thiamine and vitamin B12. These vitamins may be obtained commercially, for example from Sigma (Saint Louis, Missouri).

Inorganic salt ingredients which may be used in the media include one or more calcium salts (e.g., CaCl₂)), Fe(NO₃)₃, KCl, one or more magnesium salts (e.g., MgCl2 and/or MgSO₄), one or more manganese salts (e.g., MnCl₂), NaCl, NaHCO₃, N₂HPO₄, and ions of the trace elements selenium, vanadium, zinc and copper. These trace elements may be provided in a variety of forms, preferably in the form of salts such as Na₂SeO₃, NH₄VO₃, ZnSO₄ and CuSO₄. These inorganic salts and trace elements may be obtained commercially, for example from Sigma (Saint Louis, Missouri).

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are typically those previously used with the host cell selected for expression, and will be apparent to the ordinarily skilled artisan. Exemplary culture conditions are provided in M. Takagi and K. Ueda, “Comparison of the optimal culture conditions for cell growth and tissue plasminogen activator production by human embryo lung cells on microcarriers”, Biotechnology, (1994), 41, 565-570: H. J. Morton, “A survey of commercially available tissue culture media”, In Vitro (1970), 6(2), 89-108: J. Van der Valk, et al., (2010), “Optimization of chemically defined cell culture media-replacing fetal bovine serum in mammalian in vitro methods,” Toxicology in vitro, 24(4), 1053-1063; R. J. Graham et al., “Consequences of trace metal variability and supplementation on Chinese hamster ovary (CHO) cell culture performance: A review of key mechanisms and considerations”, BIotechnol. Bioeng. (2019), 116(12), 3446-3456: S. Janoschek et al., A protocol to transfer a fed-batch platform process into semi-perfusion mode: The benefit of automated small-scale bioreactors compared to shake flasks as scale-down model”, Biotechnol. Prog., (2019), 35(2), e2757; and M. Kuiper et al., “Repurposing fed-batch media and feeds for highly productive CHO perfusion processes”, Biotechnology Progress, 15 Apr. 2019, https://doi.org/10.1002/btpr.2821: all of which are incorporated by reference herein in their entirety.

Cell culture intended for the production of recombinant protein may comprise eukaryotic cells encoding the recombinant protein. The recombinant protein can be produced by growing cells which express the products of interest under a variety of cell culture conditions. For instance, cell culture procedures for the large or small-scale production of proteins are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode.

In a fed batch culture, the host cells (which may be eukaryotic cells, e.g. mammalian host cells) and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process: See for e.g.: F. Li, N. Vijayasankaran, A. Shen, R. Kiss, A. Amanullah: MAbs 2 (5), 466-479. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

Fed batch or continuous cell culture conditions are typically devised to enhance growth of the eukaryotic cells (e.g. mammalian cells) in the growth phase of the cell culture. In the growth phase cells are grown under conditions and for a period of time that is maximized for growth. Culture conditions, such as temperature, pH, dissolved oxygen (dO₂) and the like, are those used with the particular host and will be apparent to the ordinarily skilled artisan.

### Products

In the methods of optimizing sialic acid for a recombinant polypeptide of the disclosure, the recombinant polypeptide may be any recombinant polypeptide product of interest that comprises sialic acids. The methods of the present disclosure may be used for the production of polypeptides, e.g., mammalian polypeptides. Non-limiting examples of such polypeptides include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. The cells, and/or cell lines, and/or methods of the present disclosure are typically not specific to the molecule, e.g., antibody, that is being produced.

In the methods of the present disclosure, the recombinant polypeptides for which sialic acid content is optimized may be an antibodies, including therapeutic and diagnostic antibodies or antigen-binding fragments thereof. The antibodies can be, but are not limited to, monospecific antibodies (e.g., antibodies consisting of a single heavy chain sequence and a single light chain sequence, including multimers of such pairings), multispecific antibodies and antigen-binding fragments thereof. For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof.

### Multispecific Antibodies

An antibody may be a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). The multispecific antibody may have three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305:537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004): cross-linking two or more antibodies or fragments (see, e.g., U.S. Pat. No. 4,676,980, and Brennan et al., Science, 229:81 (1985)): using leucine zippers to produce bi-specific antibodies (see, e.g., Kostelny et al., J. Immunol., 148(5):1547-1553 (1992) and WO 2011/034605): using the common light chain technology for circumventing the light chain mis-pairing problem (see, e.g., WO 98/50431): using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147:60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539.

Multispecific antibodies may also be provided in an asymmetric form with a domain crossover in one or more binding arms of the same antigen specificity, i.e., by exchanging the VH/VL domains (see, e.g., WO 2009/080252 and WO 2015/150447), the CHI/CL domains (see, e.g., WO 2009/080253) or the complete Fab arms (see, e.g., WO 2009/080251, WO 2016/016299, also see Schaefer et al, PNAS, 108 (2011) 1187-1191, and Klein at al., MAbs 8 (2016) 1010-20). A multispecific antibody may comprise a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bäuerle, Exp Cell Res 317, 1255-1260 (2011)); diabodies (Holliger et al., Prot. Eng. 9, 299-305 (1996)) and derivatives thereof, such as tandem diabodies (“TandAb”: Kipriyanov et al., J Mol Biol 293, 41-56 (1999)): “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8)(2016) e1203498.

### Antibody Fragments

An antibody for which sialic acid content may be optimized in accordance with methods provided herein may be or comprise an antibody fragment. For example, but not by way of limitation, the antibody fragment may be a Fab, Fab′, Fab-SH or F(ab)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CHI domain including one or more cysteines from the antibody hinge region. Fab′-SH are Fab′ fragments in which the cysteine residue(s) of the constant domains bear a free thiol group. Pepsin treatment yields an F(ab)₂ fragment that has two antigen-binding sites (two Fab fragments) and a part of the Fc region. For discussion of Fab and F(ab)₂ fragments comprising salvage receptor binding epitope residues and having increased in vivo half-life, see U.S. Pat. No. 5,869,046.

The antibody fragment may be a diabody, a triabody or a tetrabody. “Diabodies” are antibody fragments with two antigen-binding sites that can be bivalent or bispecific. See, for example, EP 404,097: WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

The antibody fragment may be a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CHI-linker-VL-CL, b) VL-CL-linker-VH-CHI, c) VH-CL-linker-VL-CHI or d) VL-CHI-linker-VH-CL. In particular, said linker may be a polypeptide of at least 30 amino acids, preferably between 32 and 50 amino acids. Said single chain Fab fragments are stabilized via the natural disulfide bond between the CL domain and the CHI domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

The antibody fragment may be a single-chain variable fragment (scFv). A “single-chain variable fragment” or “scFv” is a fusion protein of the variable domains of the heavy (VH) and light chains (VL) of an antibody, connected by a linker. In particular, the linker may be a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994): see also WO 93/16185; and U.S. Pat. Nos. 5,571,894 and 5,587,458.

The antibody fragment may be a single-domain antibody. “Single-domain antibodies” are antibody fragments comprising all or a portion of the heavy chain variable domain or all or a portion of the light chain variable domain of an antibody. A single-domain antibody may be a human single-domain antibody (Domantis, Inc., Waltham, MA: see, e.g., U.S. Pat. No. 6,248,516 B1).

Antibody fragments may be made by various techniques, including but not limited to proteolytic digestion of an intact antibody.

### Chimeric and Humanized Antibodies

An antibody for which sialic acid content may be optimized in accordance with methods provided herein may be a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

A chimeric antibody may be a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989): U.S. Pat. Nos. 5,821,337, 7,527,791, 6,982,321, and 7,087,409; Kashmiri et al., Methods 36:25-34 (2005) (describing specificity determining region (SDR) grafting): Padlan, Mol. Immunol. 28:489-498 (1991) (describing “resurfacing”): Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)): framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)): human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

An antibody for which sialic acid content may be optimized in accordance with methods provided herein may be a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology: U.S. Pat. No. 5,770,429 describing HUMAB® technology: U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies may also be made by hybridoma-based methods. Human myeloma and mouse-human heteromyeloma cell lines for the production of human monoclonal antibodies have been described. (See, e.g., Kozbor J. Immunol., 133:3001 (1984): Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### Target Molecules

Non-limiting examples of molecules that may be targeted by an antibody with optimized sialic acid content produced by the methods disclosed herein include soluble serum proteins and their receptors and other membrane bound proteins (e.g., adhesins). In certain examples, the antibody is capable of binding to one, two or more cytokines, cytokine-related proteins, and cytokine receptors. The cytokine receptors may be selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (aFGF), FGF2 (βFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNAI, IFNA2, IFNA4, IFNA5, IFNA6, IFNA7, IFN81, IFNG, IFNWI, FEL1, FEL1 (EPSELON), FEL1 (ZETA), IL IA, IL 1B, IL2, IL3, IL4, IL5, IL6, IL7, IL8, IL9, ILI 0, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 1IRA, IL12RB1, IL12RB2, IL13RA1, IL13RA2, IL15RA, IL17R, IL18R1, IL20RA, IL2IR, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

An antibody with optimized sialic acid content produced according to a method disclosed herein may be capable of binding to a chemokine, chemokine receptor, or a chemokine-related protein selected from the group consisting of CCLI (1-309), CCL2 (MCP-1/MCAF), CCL3 (MIP-Iα), CCL4 (MIP-Iβ), CCL5 (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CRI (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8RB), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In certain examples, the recombinant polypeptide with optimized sialic acid content obtained according to the methods of the present invention is an antibody (e.g., a multispecific antibody such as a bispecific antibody) that is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (Bcell-activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3β); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRP/RA); CCR3 (CKR/CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79u, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDGI; EFNA1; EFNA3; EFNB2; EGF; EGFR; ELAC2; ENG; ENOl; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR; FGFR3; FIGF (VEGFD); FELl (EPSILON); FILl (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISSIR; GPR54; HOT7T175; AXOR12); GPR81 (FKSG80); GPR172A (G protein-coupled receptor 172A; GPCR41; FLJ11856; D15Ertd747e); GRCCIO (C10); GRP; GSN (Gelsolin); GSTP1; HAVCR2; HDAC4; HDAC5; HDAC7A; HDAC9; HGF; HIF1A; HOPI; histamine and histamine receptors; HLA-A; HLA-DOB (Beta subunit of MHC class II molecule (Ia antigen); HLA-DRA; HM74; HMOXI; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-l; IL10; IL10RA; IL1ORB; IL11; IL1 IRA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL20Ru; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAKI; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MK167; (Ki-67); MMP2; MMP9; MPF (MPF, MSLN, SMR, megakaryocyte potentiating factor, mesothelin); MS4A1; MSG783 (RNF124, hypothetical protein FLJ20315); MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4Ai; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-Li; PD-L2; PD-1; POGFA; POGFB; PECAMI; PF4 (CXCL4); PGF; PGR; phosphacan; PIAS2; PIK3CG; PLAU (uPA); PLG; PLXDCi; PMEL17 (silver homolog; SILV; DI2S53E; PMEL17; SI; SIL); PPBP (CXCL7); PPID; PRI; PRKCQ; PRKDI; PRL; PROC; PROK2; PSAP; PSCA hlg (2700050C12Rik, C530008016Rik, RIKEN cDNA 2700050Ci2, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGBID2 (lipophilin B); SCGB2A1 (mammaglobin2); SCGB2A2 (mammaglobin 1); SCYEI (endothelial Monocyte-activating cytokine); SDF2; Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B); SERPINAI; SERPINA3; SERPINB5 (maspin); SERPINEI(PAI-1); SERPDMFI; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAPI, STAMPI, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSFlA; TNFRSFlB; TNFRSF21; TNFRSF5; TNFRSF6 (Fas); TNFRSF7; TNFRSF8; TNFRSF9; TNFSF1O (TRAIL); TNFSF11 (TRANCE); TNFSF12 (AP03L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, FLJ20041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3); VEGF; VEGFB; VEGFC; versican; VHL C5; VLA-4; XCL1 (lymphotactin); XCL2 (SCM-1b); XCRI(GPR5/CCXCRI); YY1; and ZFPM2.

### Optimizing Sialic Acid Content

It is desirable to optimize the level of sialic acid in many recombinant polypeptides. See, for instance, S. Weikert, et al., “Engineering Chinese hamster ovary cells to maximize sialic acid content of recombinant glycoproteins”, Nature Biotechnology (1999), vol. 17(11), 1116-1121. Antibodies, for example, comprise sialic acid. In many applications that use antibodies, it is important that the antibodies have the appropriate levels of sialic acid, which may be reflected by the specification for an antibody product specifying that antibody should be present at within a given range. See for e.g. Engineering Chinese hamster ovary cells to maximize sialic acid content of recombinant glycoproteins, S. Weikert, D. Papac, J. Briggs, D. Cowfer, S. Tom, M. Gawlitzek, J. Lofgren, S. Mehta, V. Chisholm, N. Modi, S. Eppler, K. Carroll, S. Chamow, D. Peers, P. Berman & L. Krummen; Nature Biotechnology volume 17, pages 1116-1121 (1999).

Recombinant polypeptides that comprise sialic acids are typically produced in cell culture, using a mammalian cell line engineered to express the recombinant polypeptide. We have observed that the level of sialic acid content often decreases at later stages of the culture. Such a decease may result is a recombinant polypeptide product that has a sub-optimal sialic acid level. For example, it may result in a therapeutic antibody that cannot be used, as its sialic acid level is below the specification. It is therefore desirable to have a method that will maximize the chance that the recombinant polypeptide is isolated from the cell culture while it has an optimal level of sialic acid (for example a level of sialic acid in mol per mol polypeptide that falls within the specification).

We have determined that cell viability is predictive of the level of sialic acid, for example for antibodies. In particular, we have determined that cell viability as determined at a relatively early stage of the cell culture (e.g. on one of days 6 to 11 or 8 to 11) is predictive of optimal level of sialic acid at a relatively later stage of the cell culture (e.g. on one or more of days 9 to 14). In view of this, we have developed methods for optimizing sialic acid content for a recombinant polypeptide. As these methods may allow the cell culture to continue for longer in some instances, this may also result in a relatively high titer.

An aspect of the present invention provides a method of optimizing sialic acid content for a recombinant polypeptide. The method comprises culturing a cell-line engineered to express the recombinant polypeptide under conditions promoting production of the recombinant polypeptide; monitoring cell viability of the cultured cell-line on at least one of days 6, 7, 8, 9, 10, 11 or 12 of the culturing. If the cell viability is at or below a threshold level, the method also comprises stopping the culture and isolating the recombinant polypeptide after a first predetermined additional time. If the cell viability is above the threshold level, the method also comprises stopping the culture and isolating the recombinant polypeptide after a second predetermined additional time. The second predetermined additional time is at least about 10 hours longer (e.g. 12 hours longer) than the first predetermined time.

In embodiments, the monitoring the cell viability is performed on at least one of days 6, 7, 8, 9, 10, or 11. For example, the monitoring the cell viability may performed on at least one of days 7, 8, 9, or 10. The monitoring the cell viability may performed on at least one of days 8, 9, or 10. The monitoring the cell viability may performed on day 8. The monitoring the cell viability may performed on day 9. The monitoring the cell viability may performed on day 10.

In embodiments, the threshold level is at least about 65% cell viability. For example, the threshold level may be at least about 70% cell viability. The threshold level may be at least about 75% cell viability. The threshold level may be at least about 80% cell viability. The threshold level may be at least about 85% cell viability.

The threshold level may be at least about 90% cell viability. The threshold level may be at least about 95% cell viability.

In embodiments, the threshold level is selected from about 65%, about 70%, about 75%, about 80%, about 85%, about 90%, or about 95% cell viability. For example, the threshold level may be selected from about 75%, about 80%, about 85%, or about 90% cell viability.

The duration of each of the first predetermined additional time and second predetermined additional time may be determined from the time point when the monitoring the cell viability is performed. Where the monitoring comprises sampling followed by on-line or off line analysis, the said additional time is determined from the time of sampling. The duration of the second predetermined additional time may also (or instead) be specified by how much longer it is than the first predetermined additional time.

In embodiments, the first predetermined additional time is selected such that the culturing will be performed for a total time of at least about 8 days. The first predetermined additional time may be selected such that the culturing will be performed for a total time of at least about 9 days. For example, the first predetermined additional time may be selected such that the culturing will be performed for a total time of at least about 10 days. The first predetermined additional time may be selected such that the culturing will be performed for a total time of at least about 11 days. The first predetermined additional time may be selected such that the culturing will be performed for a total time of at least about 12 days.

In embodiments, the first predetermined additional time comprises a time selected from the range of from about 0.5 days to about 4 days. For example, the first predetermined additional time may comprise a time selected from the range of from about 0.5 days to about 3 days; e.g. the first predetermined additional time may comprise a time selected from the range of from about 0.5 days to about 2 days. The first predetermined additional time may comprise a time selected from the range of from about 1 day to about 3 days; e.g. the first predetermined additional time may comprise a time selected from the range of from about 1 day to about 2 days.

In embodiments, the first predetermined additional time comprises at least about 10 hours. The first predetermined additional time may comprise at least about 12 hours. The first predetermined additional time may comprise at least about 18 hours. The first predetermined additional time may comprise at least about 24 hours.

The first predetermined additional time may comprise at least about 36 hours. The first predetermined additional time may comprise at least about 48 hours.

In embodiments, the first predetermined additional time comprises not more than about 72 hours. The first predetermined additional time may comprise not more than about 60 hours. The first predetermined additional time may comprise not more than about 48 hours. The first predetermined additional time may comprise not more than about 36 hours. The first predetermined additional time may comprise not more than about 24 hours.

In embodiments, the second predetermined additional time is at least about 1 day longer than the first predetermined additional time. For example, the second predetermined additional time may be at least about 1.5 days longer than the first predetermined additional time. The second predetermined additional time may be at least about 2 days longer than the first predetermined additional time. The second predetermined additional time may be at least about 2.5 days longer than the first predetermined additional time. The second predetermined additional time may be at least about 3 days longer than the first predetermined additional time. The second predetermined additional time may be at least about 3.5 days longer than the first predetermined additional time. The second predetermined additional time may be at least about 4 days longer than the first predetermined additional time. The second predetermined additional time may be at least about 4.5 days longer than the first predetermined additional time.

In embodiments, the second predetermined additional time is about 1 day longer than the first predetermined additional time. The second predetermined additional time may be about 1.5 days longer than the first predetermined additional time. The second predetermined additional time may be about 2 days longer than the first predetermined additional time. The second predetermined additional time may be about 2.5 days longer than the first predetermined additional time. The second predetermined additional time may be about 3 days longer than the first predetermined additional time. The second predetermined additional time may be about 3.5 days longer than the first predetermined additional time. The second predetermined additional time may be about 4 days longer than the first predetermined additional time. The second predetermined additional time may be about 4.5 days longer than the first predetermined additional time.

In embodiment, the second predetermined additional time may comprise a time selected from the range of from about 1 days to about 6.5 days. For example, the second predetermined additional time may comprise a time selected from the range of from about 1.5 days to about 6.5 days. The second predetermined additional time may comprise a time selected from the range of from about 1.5 days to about 5.5 days. The second predetermined additional time may comprise a time selected from the range of from about 1.5 days to about 4.5 days. The second predetermined additional time may comprise a time selected from the range of from about 1.5 days to about 3.5 days. The second predetermined additional time may comprise a time selected from the range of from about 2 days to about 6 days. The second predetermined additional time may comprise a time selected from the range of from about 2 days to about 5 days. The second predetermined additional time may comprise a time selected from the range of from about 2 days to about 4 days. The second predetermined additional time may comprise a time selected from the range of from about 2 days to about 3 days.

In embodiments, the second predetermined additional time comprises at least about 2 days. The second predetermined additional time may comprise at least about 3 days. The second predetermined additional time may comprise at least about 4 days. The second predetermined additional time may comprise at least about 5 days.

In embodiments, the second predetermined additional time comprises not more than about 6.5 days. The second predetermined additional time may comprise not more than about 5.5 days. The second predetermined additional time may comprise not more than about 4.5 days. The second predetermined additional time may comprise not more than about 3.5 days. The second predetermined additional time may comprise not more than about 2.5 days.

In embodiments, the recombinant polypeptide comprises at least two N-linked glycan sites. For example, the recombinant polypeptide may comprise at least three N-linked glycan sites. The recombinant polypeptide may comprise at least four N-linked glycan sites. The recombinant polypeptide may comprise at least five N-linked glycan sites. The recombinant polypeptide may comprise at least six N-linked glycan sites.

In embodiments, the recombinant polypeptide comprises two to twelve N-linked glycan sites. The recombinant polypeptide may comprise two to ten N-linked glycan sites. The recombinant polypeptide may comprise four to twelve N-linked glycan sites. The recombinant polypeptide may comprise four to ten N-linked glycan sites. The recombinant polypeptide may comprise six to twelve N-linked glycan sites. The recombinant polypeptide may comprise six to ten N-linked glycan sites.

In embodiments, the recombinant polypeptide may comprise 2, 3, 4, 5, 6, 7, 8, 9, 10, 11, or 12 N-linked glycan sites. The recombinant polypeptide may comprise 2, 3, 4, 5, 6, 7, 8, 9, or 10 N-linked glycan sites. The recombinant polypeptide may comprise 4, 5, 6, 7, 8, 9, or 10 N-linked glycan sites. The recombinant polypeptide may comprise 6, 7, 8, 9, or 10 N-linked glycan sites.

In embodiments, the recombinant polypeptide is an antibody, an antigen, an enzyme, or a vaccine. The antibody may be a multispecific antibody (e.g. a bispecific antibody) or antigen-binding fragment thereof. The antibody may be a multispecific antibody, such as a bispecific antibody. The antibody may be an antigen-binding fragment of an antibody or multispecific (e.g. bispecific) antibody. The antibody may comprise or consist of a single heavy chain sequence and a single light chain sequence or antigen-binding fragments thereof. The antibody may comprise or consist of a single heavy chain sequence and a single light chain sequence. The antibody may comprise or consist of antigen-binding fragments of a single heavy chain sequence and a single light chain sequence. The antibody may comprise a chimeric antibody, a human antibody or a humanized antibody. The antibody may comprise a chimeric antibody. The antibody may comprise a human antibody. The antibody may comprise a humanized antibody. The antibody may comprise a monoclonal antibody.

In embodiments, the recombinant polypeptide is a fusion protein, for example comprising an antibody (such as a multispecific antibody, e.g. a bispecific antibody) or an antigen-binding fragment thereof. The recombinant polypeptide may be a fusion protein comprising an antibody. The recombinant polypeptide may be a fusion protein comprising an antigen-binding fragment of an antibody. The antibody may comprise or consist of a single heavy chain sequence and a single light chain sequence or antigen-binding fragments thereof. The antibody may comprise or consist of a single heavy chain sequence and a single light chain sequence. The antibody may comprise or consist of antigen-binding fragments of a single heavy chain sequence and a single light chain sequence. The antibody may comprise a chimeric antibody, a human antibody or a humanized antibody. The antibody may comprise a chimeric antibody. The antibody may comprise a human antibody. The antibody may comprise a humanized antibody. The antibody may comprise a monoclonal antibody.

In embodiments, the recombinant polypeptide is an Fc fusion protein. The Fc fusion protein may be or comprise a multispecific (e.g. a bispecific) antibody. The Fc fusion protein may be or comprise a cytokine Fc fusion protein.

In embodiments, the cell line is an animal cell line. For example, the cell line may be a mammalian cell line. The cell line may be a CHO cell line or an NSO cell line. The cell line may be a CHO cell line. For example, the cell line may be a CHO cell line selected from a CHO K1 cell line, a CHO K1SV cell line, a DG44 cell line, a DUKXB-11 cell line, a CHOKIS cell line, or a CHO KIM cell line, or their derivatives. In embodiments, the CHO cell line may be a targeted gene integration (TI) -generated CHO cell line. The cell line may be an NSO cell line. For example, the cell line may be an NSO cell line.

In embodiments, the cell line is cultured in a cell culture medium. The cell line may be cultured under fed-batch culture conditions, or perfusion culture conditions. For example, the cell line may be cultured under fed-batch culture conditions. The fed-batch culture conditions may comprise intensified fed-batch culture conditions. For example, the cell line may be cultured under perfusion culture conditions. The perfusion culture conditions may comprise semi-continuous perfusion or continuous perfusion.

In embodiments, the isolated recombinant polypeptide comprises sialylation at a level of about 5 mol per mol recombinant polypeptide to about 20 mol per mol recombinant polypeptide. The isolated recombinant polypeptide may comprise sialylation at a level of about 7 mol per mol recombinant polypeptide to about 15 mol per mol recombinant polypeptide. The isolated recombinant polypeptide may comprise sialylation at a level of about 8 mol per mol recombinant polypeptide to about 12 mol per mol recombinant polypeptide.

### EXAMPLES

### Example 1—Recombinant Cytokine Fc Fusion Protein

### Materials and Methods

Data shown in this paper are from experiments that were performed using a recombinant Chinese hamster ovary (CHO) cell line that is genetically engineered to secrete a recombinant Fc fusion protein with eight N-glycosylation sites.

Cryogenically frozen ampules of this cell line were thawed into an in-house developed and proprietary chemically-defined medium and cultivated in shaker flasks for at least two weeks at 37° C. in humidified incubators with 5% CO2 before use for any bioreactor experiments. After the cells exhibited good growth characteristics and high viability, they were scaled up for production culture experiments in 2-liter stirred suspension bioreactors (Applikon, Foster City, CA). Production cultures were usually inoculated at approximately 1.0 million viable cells/mL in a chemically-defined basal medium, which was also developed in-house. The culture was exposed to the production culture medium through two or three inoculum stage cultures, each lasting 3 days. The production cultures were operated in the fed-batch mode, with a concentrated proprietary chemically-defined nutrient medium fed to the culture on days 3 at 20% of the culture volume. The concentration of glucose was analyzed every day and if the glucose concentration fell below a certain threshold, it was replenished from a stock solution of glucose to prevent glucose depletion.

Reactors were equipped with calibrated dissolved oxygen, pH and temperature probes. Dissolved oxygen was controlled on-line through sparging with air and/or oxygen. pH was controlled through additions of CO₂ or Na₂CO₃. Antifoam was added to the cultures as needed. The cultures were controlled to maintain the pH at 7.0, temperature at 37° C. from days 0 through 3 and then at 33° C. after day 3, agitation at 275 rpm, and dissolved oxygen level at 30% of air saturation. Viable cell density (VCC), viability, offline pH, osmolality and metabolite concentrations were determined daily using a NovaFlex (Nova Biomedical, Waltham, MA). Graduated centrifuge tubes (Kimble Science Products, Fullerton, CA) were used to measure packed cell volume (PCV) after centrifugation of cell suspension for 10 min at 830 g. PCV was expressed as a percentage of the total culture volume. The supernatant of the culture was also collected daily by centrifuging 1 mL of cell culture fluid and was used for product titer determination using high performance liquid chromatography.

### Measurement of Sialic Acid Content

For all cultures that were performed for this study, the cell culture fluid at the end of the culture duration (typically 12 days) was harvested by centrifugation. The monoclonal antibody in the harvested cell culture fluid was purified using protein A affinity chromatography. The sialic acid reversed phase-ultra high performance liquid chromatography (RP-UHPLC) method is used to determine N-acetylneuraminic acid (NANA) content. Samples are acid hydrolyzed followed by derivatization with o-phenylenediamine (OPD). Analysis is by C-18 RP-UHPLC with fluorescence detection. The concentration is calculated from an external calibration curve and is reported as moles of NANA per mole of the protein. To characterize the impact of culture duration on sialic acid content, cell culture fluid samples from earlier culture durations were also analyzed in this manner to measure sialic acid content.

### Results and Discussion

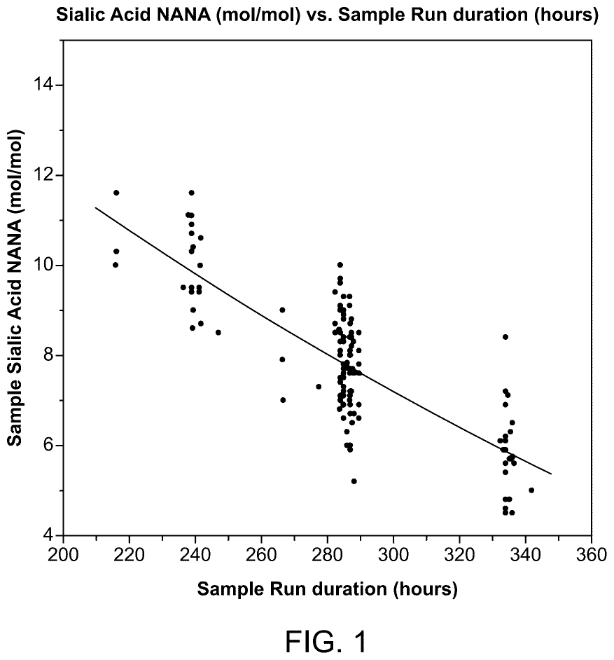

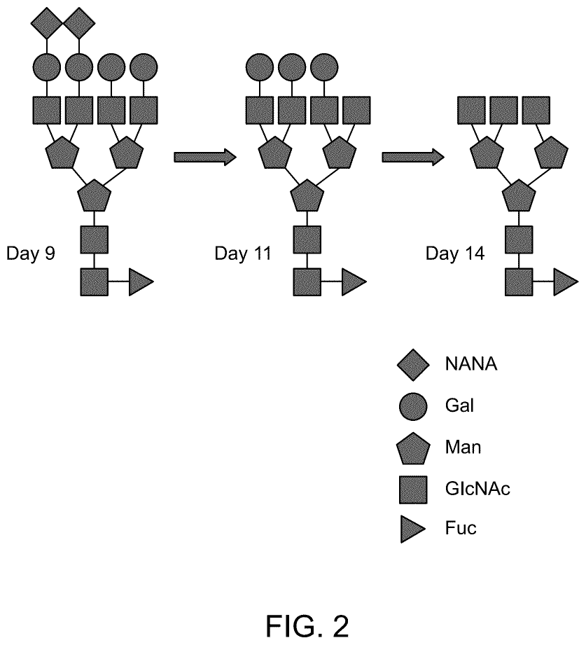

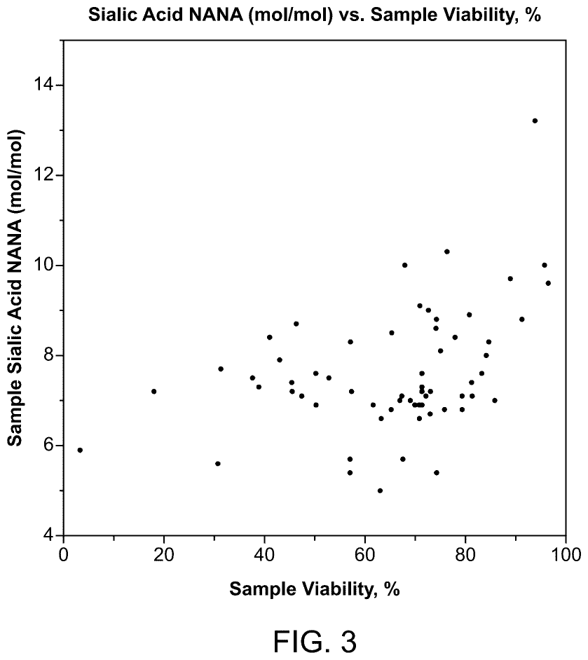

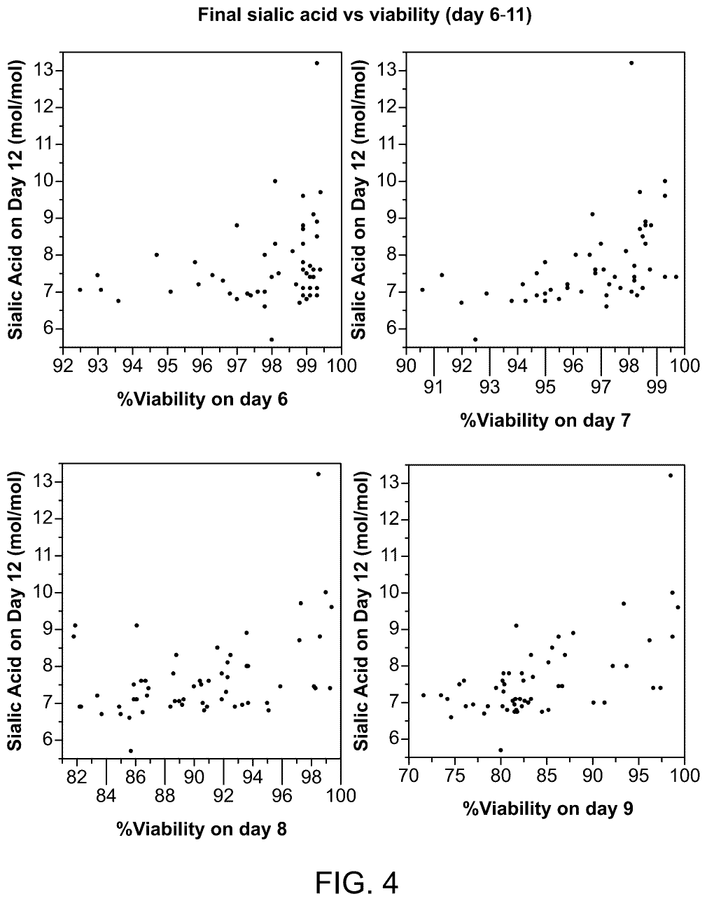

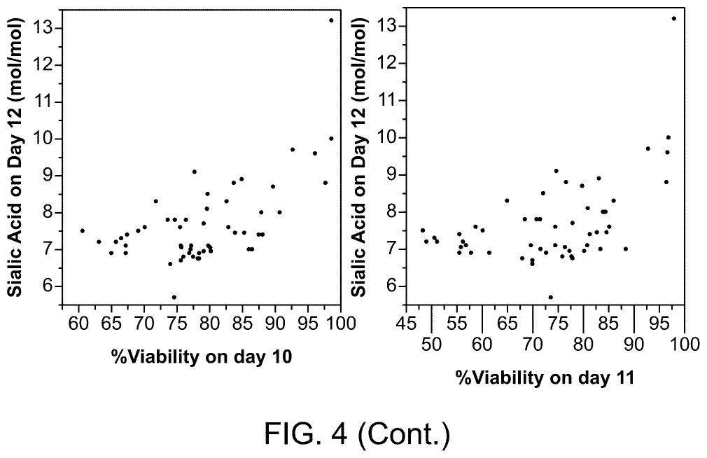

The results obtained are illustrated in FIGS. 1, 3 and 4. FIG. 1 shows that sialic acid content measured during the course of the cell culture run declines with increasing culture duration. FIG. 3 shows that the sialic acid content measured at the end of the culture (day 12) is correlated with the viability of the culture measured on the day i.e. greater the culture viability greater is the sialic acid content. FIG. 4 shows that viability of the culture measured earlier in the duration (day 10) is correlated sialic acid content measured on the final day of the culture (day 12) i.e. the sialic acid content at the end of the culture can be predicted by measurements of viability from earlier in the culture. Hence, modulating the total duration of the culture based on the measurements of cell viability earlier in the culture could be used to control the level of sialic acid content at the end of the culture.

### Example 2—Recombinant Monoclonal Antibody

Recombinant Chinese hamster ovary (CHO) cell line that was genetically engineered to secrete a monoclonal antibody were prepared and cultured in a similar method to that described for Example 1.

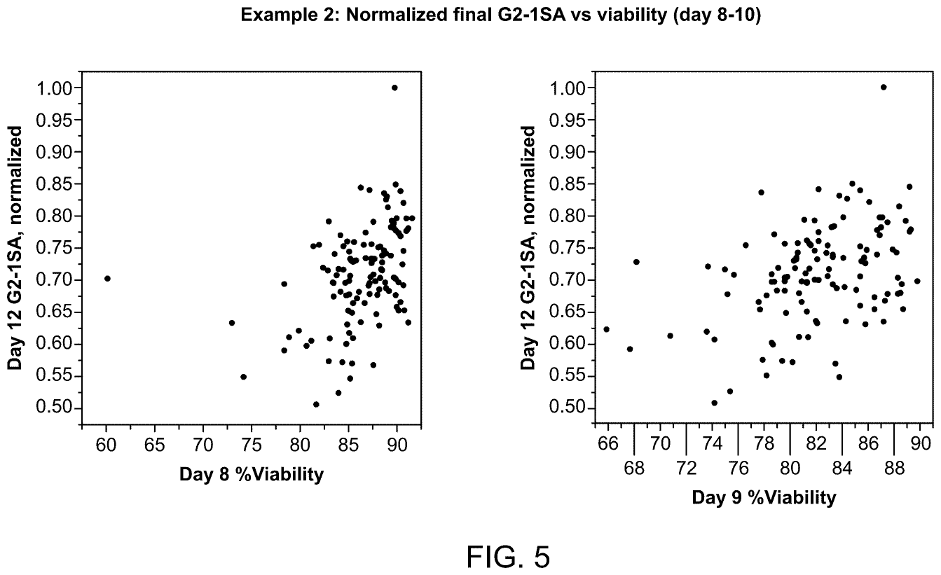

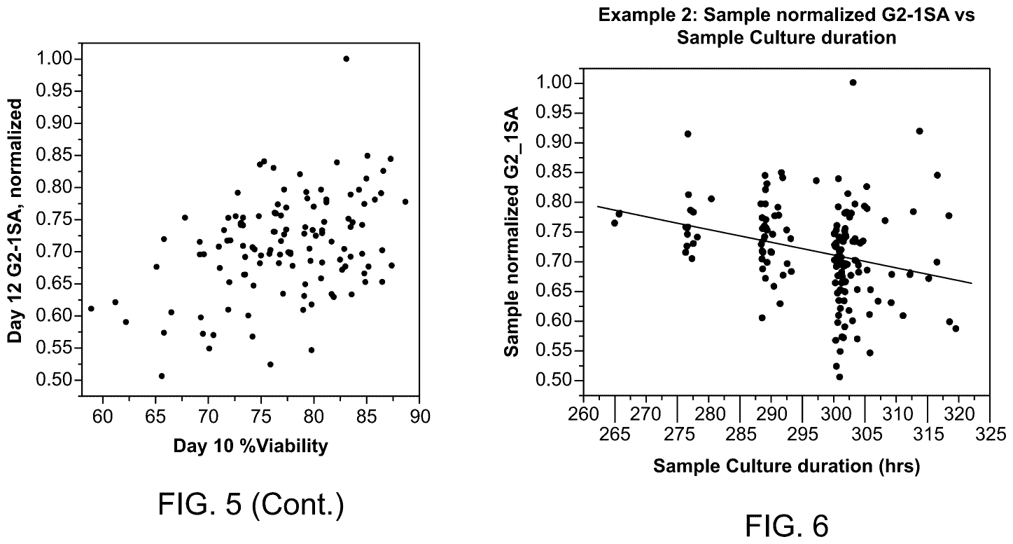

The viability of the cell culture was measured on each of days 8, 9, 10, as well as on the final day of the cell culture (day 12). The final levels of a sialylated species (G2-1SA) that is present in the optimally sialylated antibody was found to correlate with the viability on days 8, 9, 10. FIG. 5 shows the correlation between the viability measured on days 8, 9, 10 of the culture and the levels of a sialylated glycan species (G2-1SA) at the end of the culture (day 12). G2-1SA is normalized vs the highest reported G2-1SA in the sample set of optimally sialylated antibody (i.e. normalized G2-1SA of 0.50 is 50% of the highest reported G2-1SA value in the data set). FIG. 6 also shows a correlation between normalized G2-1SA and culture duration, therefore this example also shows that viability measurements earlier in the culture may be used to control the final levels of sialylation at the end of the culture, by changing the culture duration based on the earlier viability measurement.

What is claimed is:

1. A method of optimizing sialic acid content for a recombinant polypeptide, comprising:
culturing a cell-line engineered to express the recombinant polypeptide under conditions promoting production of the recombinant polypeptide;
monitoring cell viability of the cultured cell-line on at least one of days 6, 7, 8, 9, 10, or 11 of the culturing; and
if the cell viability is at or below a threshold level, stopping the culture and isolating the recombinant polypeptide after a first predetermined additional time, or
if the cell viability is above the threshold level, stopping the culture and isolating the recombinant polypeptide after a second predetermined additional time;
wherein the second predetermined additional time is at least about 12 hours longer than the first predetermined time.

2. The method of claim 1, wherein the monitoring the cell viability is performed on at least one of days 7, 8, 9, 10, or 11.

3. The method of claim 1, wherein the monitoring the cell viability is performed on at least one of days 8, 9, 10, or 11.

4. The method of claim 1 or claim 2, wherein the monitoring the cell viability is performed on at least one of days 8, 9, or 10.

5. The method of any preceding claim, wherein the threshold level is at least about 65% cell viability.

6. The method of any preceding claim, wherein the threshold level is at least about 70% cell viability.

7. The method of any preceding claim, wherein the threshold level is at least about 75% cell viability.

8. The method of any preceding claim, wherein the threshold level is at least about 80% cell viability.

9. The method of any preceding claim, wherein the threshold level is at least about 85% cell viability.

10. The method of any preceding claim, wherein the threshold level is at least about 90% cell viability.

11. The method of any preceding claim, wherein the threshold level is at least about 95% cell viability.

12. The method of any of claims 1 to 3, wherein the threshold level is selected from about 65%, about 70%, about 75%, about 80%, about 85%, about 90% or about 95% cell viability.

13. The method of any preceding claim, wherein the first predetermined additional time is selected such that the culturing will be performed for a total time of at least about 8 days, optionally for at least about 10 days, further optionally for at least about 11 days.

14. The method of any preceding claim, wherein the first predetermined additional time comprises a time selected from the range of from about 0.5 days to about 4 days.

15. The method of any preceding claim, wherein the second predetermined additional time is at least about 1.5, at least about 2, at least about 2.5, at least about 3, at least about 3.5, or at least about 4 days longer than the first predetermined additional time.

16. The method of any preceding claim, wherein the second predetermined additional time comprises a time selected from the range of from about 1.5 days to about 6 days.

17. The method of any preceding claim, wherein the recombinant polypeptide comprises at least two N-linked glycan sites.

18. The method of any preceding claim, wherein the recombinant polypeptide is an antibody, an antigen, an enzyme, or a vaccine.

19. The method of claim 14, wherein the antibody is a multispecific antibody or antigen-binding fragment thereof.

20. The method of claim 14 or claim 15, wherein the antibody consists of a single heavy chain sequence and a single light chain sequence or antigen-binding fragments thereof.

21. The method of any of claims 14-16, wherein the antibody comprises a chimeric antibody, a human antibody or a humanized antibody.

22. The method of any of claims 14-17, wherein the antibody comprises a monoclonal antibody.

23. The method of any preceding claim, wherein the recombinant polypeptide is a fusion protein, optionally comprising an antibody or an antigen-binding fragment thereof.

24. The method of claim 19, wherein the recombinant polypeptide is an Fc fusion protein.

25. The method of any preceding claim, wherein the cell line is a mammalian cell line.

26. The method of any preceding claim, wherein the cell line is a CHO cell line or an NSO cell line; optionally a CHO KI cell line, a CHO KISV cell line, a DG44 cell line, a DUKXB-11 cell line, a CHOKIS cell line, or a CHO KIM cell line, or a targeted gene integration (TI)—generated CHO cell line, or their derivatives.

27. The method of any preceding claim, wherein the cell line is cultured in a cell culture medium.

28. The method of any preceding claim, wherein the cell line is cultured under fed-batch culture conditions, or perfusion culture conditions.

29. The method of claim 24, wherein the cell line is cultured under fed-batch culture conditions, optionally wherein the fed-batch culture conditions are intensified fed-batch culture conditions.

30. The method of claim 24, wherein the cell line is cultured under perfusion culture conditions, optionally wherein the perfusion culture conditions are semi-continuous perfusion or continuous perfusion.

31. The method of any preceding claim, wherein the isolated recombinant polypeptide comprises sialylation at a level of about 5 mol per mol recombinant polypeptide to about 20 mol per mol recombinant polypeptide.

32. The method of any preceding claim, wherein the isolated recombinant polypeptide comprises sialylation at a level of about 8 mol per mol recombinant polypeptide to about 12 mol per mol recombinant polypeptide.
